(19) United States
(12) Patent Application Publication
(10) Pub. No.: US 2011/0212150 A1
(43) Pub. Date: Sep. 1, 2011

(54) PROCESS FOR THE TREATMENT OF CELLULOSIC MOLDED BONES

(75) Inventors: Sigrid Redlinger, Lenzing (AT); Werner Richardt, St. Georgen (AT); Heinrich Firgo, Voecklabruck (AT)

(73) Assignee: LENZING AKTIENGESELLSCHAFT, Lenzing (AT)

(21) Appl. No.: 13/119,807

(22) PCT Filed: Aug. 27, 2009
(86) PCT No.: PCT/AT2009/000334

§ 371 Date: May 19, 2011

(30) Foreign Application Priority Data
Sep. 22, 2008 (AT) A 1471/2008

(51) Int. Cl. A61K 9/00; B05D 3/10; B32B 23/04; B32B 5/02; B32B 33/00; A61K 31/722; A61P 31/04; A61P 17/02; A61P 7/04

(52) U.S. Cl. 424/404; 427/339; 428/533; 442/59; 428/96; 424/400; 424/402; 514/55

(57) ABSTRACT

The present invention relates to a process for the treatment of a cellulosic molded body, wherein the molded body is contacted with an acid solution of a chitosan. The process according to the invention is characterized in that the chitosan has a deacetylation degree of at least 80%, a nitrogen content of at least 7 w %, preferably at least 7.5 w %, a weight average molecular weight M_{w} (D) of 10 kDa to 1000 kDa, preferably 10 kDa to 160 kDa, and a viscosity in 1 w % solution in 1 w % acetic acid at 25° C. of 1000 mPas or less, preferably 400 mPas or less, in particular preferably 200 mPas or less.

### BACKGROUND OF THE INVENTION

### Field of the Invention

The invention relates to a process for the treatment of a cellulosic molded body, in particular of cellulose fibers for textiles or non-woven fabrics.

In particular the invention relates to a process for modifying the properties of cellulosic molded bodies by means of chitosan.

Chitin and chitosan are natural, biodegradable, non-toxic, non-allergenic, bioactive and biocompatible polymers with a structure similar to that of cellulose. Chitin is gained from the shells of crustaceans, a waste material of the crab and shrimp industries. The worldwide interest in the range of use for chitin has seen an enormous increase in recent years as it is regarded as the second largest resource of natural polysaccharides beside cellulose.

Chitosan consists of poly-(1,4)-2-amino-2-desoxy-beta-D-glucose and is produced by deacetylation of chitin (poly-(1,4)-2-acetamide-2-desoxy-beta-D-glucose). For reasons of solubility—chitin is insoluble in water, organic solvents, diluted acids and bases—chitosan, which is soluble in diluted acids, aqueous methanol and glycerol, has the by far greater significance.

Areas of application for chitin and chitosan are the immobilization of cells and enzymes in biotechnology, the treatment of wounds in medicine, the use as nutritional supplement and preserving agent in the food industry, the preservation of seeds in agriculture, the use as flocculating agent and chelating agent with heavy metals in sewage systems.

However, a modification of the chitin/chitosan has to be carried out for most areas of application in order to improve the solubility in aqueous systems.

The use of chitosan in the textile industry is divided into three fields of application:
the production of 100% chitosan fibers and the production of “man-made fibers” with incorporated chitosan, respectively
the finishing and coating of textile fibers
auxiliary process agents for the textile industry

Due to their antibacterial properties and inhibitory effects on the growth of pathogenic germs, chitosan fibers are used in the field of medicine, for instance as wound coverage and surgical sutures. Chitin and chitosan, respectively, can be broken down enzymatically or hydrolytically by endogenic ferments and therefore are reabsorbable fibers. The effect of such natural polymers on the healing of wounds consists in the gradual release of N-acetyl-glucosamine, the mucopolysaccharide organization of the collagen as well as the beneficial effect on the tissue growth during wound healing (e.g., EP 0 077 098, U.S. Pat. No. 4,309,534, JP81/112937 and JP84/116418).

The disadvantage of fibers made of 100% chitosan, however, consists in that they exhibit low dry strength (chitosan fibers of Messrs. Innovative Technology Ltd., Winsford, England: titer 0.25 tex; fiber strength conditioned 9 cN/tex; fiber elongation conditioned 12.4%; chitosan fibers of Messrs. Korea Chitosan Co. LTD: fiber strength conditioned 15 cN/tex; fiber elongation conditioned 26%), that they are extremely brittle and that the wet strength amounts to merely 30% of the dry strength. Therefore, either chitosan fibers are admixed to other man-made fibers, or chitosan is already added to the spinning mass during the manufacturing process of, e.g. viscose fibers.

Viscose fibers with incorporated chitin/chitosan (in the following: “chitosan-incorporated viscose fibers”) are commercially available, e.g. under the trade names Crabyon (Messrs. Omikenshi Co) and Chitopoly (Messrs. Fuji Spinning Co.). Those fibers are produced, for instance, by dispersing chitosan or acetylated chitosan in powder form with a grain size of less than 10 μm in water in an amount of 0.5 to 2% by weight and by adding it to the viscose dope (U.S. Pat. No. 5,320,903). Thereupon, fibers are produced in accordance with the conventional viscose process or also the polynosic process.

Further manufacturing processes for chitosan-incorporated viscose fibers are described in U.S. Pat. No. 5,756,111 (complex pre- and after-dissolution processes at low temperature in order to obtain alkaline chitin-chitosan solutions to be added to the viscose solution) and in U.S. Pat. No. 5,622,666 (addition of microcrystalline chitosan and a water- and/or alkali-soluble natural polymer, e.g. sodium alginate, which can form ionic bonds with the chitosan, as a dispersion to the viscose dope) and in PCT/FI90/00292 and FI 78127 (addition of micro-crystalline chitosan to the spinning mass), respectively.

AT 8388 U describes the use of a cellulose fiber incorporating a chitosan or a chitosan salt and/or having a chitosan or a chitosan salt at its surface, in a non-woven textile and/or absorbent toiletries.

The chitosan-incorporated viscose fibers exhibit an increased dye affinity, an increased water retention value, fungicidal and odor-reducing properties, but also the low wet strength viscose fibers are known for. Since chitosan prevents the growth of bacteria harmful to the skin and eliminates allergic effects, for instance, fabrics made of Chitopoly are particularly suitable for dermatitis patients.

The drawback of all the methods described consists in that the fibers thus obtained contain very fine chitosan particles, since the chitosan is not soluble in the spinning mass.

The secondary agglomeration of the chitosan in the spinning mass or the inhomogeneous distribution, respectively, results in a deterioration of the spinning properties, spinning of fibers with low titres is extremely difficult. For that reason, it is also impossible to increase the amount of incorporated chitosan, since, in doing so, there would be an immediate loss of textile data or, already during spinning, numerous fiber breakages would occur. Furthermore, leakages of chitosan occur in the spinning bath, since chitosan is soluble in acids. For the incorporation of chitosan, additional complex steps are necessary.

In order to guarantee the effect of chitosan in the final product, there has to be incorporated an amount of at least 10 w % of chitosan in the fibers, as only then there will be existent enough chitosan at the fiber surface. The chitosan incorporated inside the fibers is inaccessible and hence not effective.

Subsequently, it also was attempted to incorporate chitosan in solvent-spun cellulose fibers produced in accordance with the amine-oxide process (so-called “Lyocell fibers”), in particular because of the high wet and dry strength of Lyocell fibers.

In DE 195 44 097, a process for the fabrication of molded bodies made from polysaccharide mixtures is described, wherein cellulose and a second polysaccharide are dissolved in an organic polysaccharide solvent mixable with water (preferably NMMO), which may also contain a second solvent.

Furthermore, in KR-A 9614022, the production of chitin-cellulose fibers, referred to as “chitulose”, is described, wherein chitin and cellulose are dissolved in a solvent from the group comprising dimethyl imidazoline/LiCl, dichloro acetate/chlorinated hydrocarbon, dimethyl acetamide/LiCl, N-methylpyrrolidone/LiCl, and yarns are produced according to the wet spinning process. NMMO is not mentioned in the claims.

In EP-A 0 883 645, among other things, the addition of chitosan to the solution as a modified compound for increasing the elasticity of wraps for foodstuff is claimed. The modifying compounds must be miscible with the cellulose/NMMO/water solution.

KR-A-2002036398 describes the incorporation of chitosan derivatives with quaternary ammonium groups, which are rather difficult to produce, into fibers.

In DE-A 100 07 794, the production of polymer compositions is described, comprising a biodegradable polymer and a material consisting of sea weeds and/or the shells of sea animals, as well as the production of molded bodies therefrom. The addition of a material made of sea weeds, sea animals in powder form, in the form of a powder suspension or in liquid form to the cellulose solution produced according to the Lyocell process is also claimed. Furthermore, the material may also be added after or during the shredding of the dry cellulose as well as at any stage of the manufacturing process. Despite the addition of the additive, the fibers exhibit the same textile-mechanical properties as they would without the additive. In the Examples, only Lyocell fibers that have a brown algae powder incorporated are described, wherein, for the production of the spinning mass, the brown algae dust, NMMO and pulp and a stabilizer are mixed and heated to 94° C.

Furthermore, in the final report “Erzeugnisse aus Polysaccharidverbunden” (Taeger, E.; Kramer, H.; Meister, F.; Vorwerg, W.; Radosta, S; TITK—Thüringisches Institut für Textil-und Kunststoff-Forschung, 1997, pp. 1-47, report no. FKZ 95/NR 036 F) it is described that chitosan is dissolved in diluted organic or inorganic acids and is then precipitated in an aqueous NMMO solution. Thus, a suspension of fine chitosan crystals is obtained in the cellulose solution, which then is spun. According to said document, the chitosan remains in the solution in the form of fine crystals even after the dissolution of the cellulose. That leads to the formation of a micro-heterogeneous two-phase system in the fiber. The strength of the fiber is low (with 10% chitosan: fiber strength conditioned 19.4 cN/tex; fiber elongation conditioned 11.5%).

WO 04/007818 proposes the incorporation of a chitosonium polymer (a chitosan salt with an inorganic or organic acid), which is soluble in the spinning solution, by means of adding to the spinning solution or a precursor thereof into the Lyocell fiber.

Alternatively to incorporation, there is provided the possibility to provide textile fabrics with chitosan in the course of their preparation and production. Applying chitosan onto already fabricated fibers or textile articles containing these, is in the following also designated as “impregnation”. A fundamental problem in this connection, however, is that the chitosan applied in this way is not fixed and may be washed out rather quickly, in this way losing its positive effects.

In order to provide a solution to this problem, there is proposed in EP 1 243 688 the use of chitosan nano-particles for the fabrication of fibers, yarns, knitted and textile fabrics. Nano-chitosans are nearly spherical firm bodies having a mean diameter in the range of 10 to 300 nm, which are arranged inbetween the fibrils due to their small particle diameter. The fabrication of nano-chitosans is realized by means of spray drying, evaporation technique or depressurizing of supercritical solutions.

WO 01/32751 describes a process for the production of nano-particular chitosan for cosmetic and pharmaceutical preparations having a particle diameter of 10 to 1,000 nm, wherein the pH of an aqueous, acid chitosan solution in the presence of a surfactant is increased until the chitosan will precipitate. Furthermore, there is described in WO 91/00298 the preparation of micro-crystalline chitosan dispersions and powders with a particle diameter of 0.1 to 50 μm, wherein the pH of an aqueous, acid chitosan solution is increased until the chitosan will precipitate.

WO 97/07266 describes the treatment of a Lyocell fiber with a 0.5% acetic chitosan solution.

WO 2004/007818 describes, apart from the incorporation of a chitosonium polymer in Lyocell fibers, also the treatment of never-dried Lyocell fibers with the solution or suspension of a chitosonium polymer. It has been shown that this process is only suitable for the treatment of never-dried Lyocell fibers.

The term “never-dried” designates the status-quo of a freshly-spun fiber that has never been subjected to a drying step.

The treatment of other fiber types than never-dried Lyocell fibers (e.g. Modal fibers and viscose fibers) is not possible with the process according to WO 2004/007818.

In the Austrian patent application A 82/2008 (not pre-published) there is described a process, wherein a cellulosic molded body is contacted with an alkaline dispersion containing non-dissolved chitosan particles.

### SUMMARY OF THE INVENTION

The present invention aims at providing a process for the treatment of cellulosic molded bodies, wherein the above mentioned problems of incorporating chitosan in fibers do not exist and which is suitable for different cellulosic fiber types, in a dried as well as never-dried state. The chitosan is to be fixed in particular at the fiber surface of cellulose regenerate fibers (Lyocell fibers, Modal fibers, viscose fibers, polynosic fibers) preferably in the fabrication process, so that the chitosan will still be existent at the final product even after a series of domestic washing processes.

This aim is reached by means of a process for the treatment of a cellulosic molded body, wherein the molded body is contacted with an acid solution of a chitosan, which is characterized in that the chitosan has a deacetylation degree of at least 80%, a nitrogen content of at least 7 w %, preferably at least 7.5%, a weight average molecular weight M_{w} (D) of 10 kDa to 1000 kDa, preferably 10 kDa to 160 kDa and a viscosity of 1 w % solution in 1 w % acetic acid at 25° C. of 1000 mPas or less, preferably 400 mPas or less, particularly preferably 200 mPas or less.

Surprisingly, there has been shown that it is possible to sustainably apply chitosan to the surface of cellulosic molded body, if the molded body is treated with an acid solution containing the above specified chitosan. In particular, there was found a surprising correlation between the viscosity of chitosan in acid solution and the amount of coating obtainable in the treatment of the molded body: The lower the viscosity of a chitosan in acid solution is, the higher (this is: significantly higher) is the obtainable amount of coating on the molded body.

In this way, there may be obtained sufficient amounts of coating with comparably little effort.

The term “solution of a chitosan” means that the chitosan is present in a completely dissolved form. This term, however, does not exclude the presence of further, optionally undissolved ingredients in the treatment liquid.

For the use in the process according to the invention, chitosans with a viscosity of 1% solution in 1% acetic acid at 25° C. of 1000 mPas or less, preferably 400 mPas or less, particularly preferably 200 mPas or less, measured with a Brookfield Viscosimeter at 30 rpm, are suitable.

Furthermore, the deacetylation degree of the chitosan is of importance, too: the higher the deacetylation degree is, the better suitable is the chitosan for a use in the process according to the invention.

Suitable chitosans may in particular have a polydispersity (ratio between weight average and number average of the molecular weight) of 2 to 4.

In the literature, there is not given a uniform definition for distinguishing between chitin and chitosan.

For the purpose of the present invention, the term “chitin” is meant to indicate a β-1,4-bound polymer of 2-acetamido-2-desoxy-D-glucose having a degree of deacetylation of 0%. Also for the purpose of the present invention, the term “chitosan” indicates an at least partially deacetylated β-1,4-bound polymer of 2-acetamido-2-desoxy-D-glucose.

The process according to the invention has the advantage in comparison to the known processes for the incorporation of chitosan that an incorporated chitosan within the molded body is not accessible. Only chitosan at the surface of the molded body may come into contact with the skin and, in this way, devolve its positive effects. In order to obtain the same amount of chitosan as in the impregnation at the surface of a molded body, there have to be used significantly larger amounts of chitosan than for incorporation.

In comparison to the use of nano-chitosan, the high production costs of nano-chitosan are in particular advantageous.

In regard to the process described in WO 2004/007818, the process according to the invention has the advantage that the therein described impregnation with an acid solution of a chitosonium polymer does not work in the treatment of never-dried viscose, Modal or polynosic fibers with subsequent vapor treatment. There are obtained only very little amounts of chitosan coating, while this process cannot be performed without the reconstruction of already existing plants.

In addition, the process according to the invention is cheaper than the process described in WO 2004/007818, as there may preferably be used cheaper types of chitosan (see further below).

### DESCRIPTION OF THE FIGURES

For a more complete understanding of the present invention, and the advantages thereof, reference is made to the following descriptions taken in conjunction with the accompanying FIGURE, in which

FIG. 1 is a graph showing the amount of chitosan coating on cellulose fibers in accordance with an exemplary embodiment of the invention.

### DETAILED DESCRIPTION OF THE INVENTION

According to a preferred embodiment of the process according to the invention, the content of chitosan particles in the solution is 0.1 to 10 w %, particularly preferable 1 to 4 w %.

The molded body that is treated according to the invention is preferably present in the form of fibers. The fibers may in particular be Lyocell fibers, Modal fibers, polynosic fibers and/or viscose fibers.

The generic name “Lyocell” was issued by the BISFA (The International Bureau for the Standardisation of Man Made Fibers) and stands for cellulose fibers, which are prepared from cellulose solutions in an organic solvent. Tertiary amine oxides, in particular N-methyl-morpholine-N-oxide (NMMO), are preferably used as solvents. A process for the production of Lyocell fibers is e.g. described in U.S. Pat. No. 4,246,221.

Viscose fibers are fibers obtained from an alkaline solution of the cellulose xanthogenate (viscose) by means of precipitation and regeneration of the cellulose.

Modal fibers are cellulose fibers characterized according to the BISFA definition by high wet strength and a high wet modulus (the force necessary to expand a fiber in its wet state by 5%).

For the treatment with the chitosan solution, the fibers may be present already in dried form, in particular as integral part of a textile article, preferably a yarn, a knitted fabric, a piece of cloth produced therewith or a non-woven article.

“Already dried” fibers are fibers which have been subjected in the course of their process of production to a drying step at least once.

Preferably, however, the fibers may be present in never-dried form. A fiber is designated as “never-dried” if it has never been subjected to a drying step in the course of its fabrication. The fibers may be present in particular in the form of a fiber fleece, as it is produced in the course of the production of Lyocell, Modal and polynosic staple fibers as an intermediary product.

This variant has the advantage that the treatment may be implemented without requiring changes or modifications in apparatuses in an existing plant for the production of Lyocell, viscose, Modal or polynosic fibers. A treatment with never-dried viscose, Modal or polynosic fibers with chitosan during the process of production has not been possible so far.

The fibers may have residual moisture of 50% to 500% before treatment.

After treatment with the chitosan solution, the molded body may be subjected to a treatment with hot vapor. In this way, there may be obtained additional fixation of the chitosan on the surface of the molded body.

In order to produce the solution, there is preferably dissolved chitosan in an inorganic or organic acid.

The acid is preferably selected from the group consisting of mono-, di- or tricarboxylic acids with 1 to 30 C-atoms, preferably lactic acid, acetic acid, formic acid, propionic acid, glycolic acid, citric acid, oxalic acid and mixtures thereof.

It has been shown that the amount of acid necessary for the dissolution of the chitosan is dependent on the deacetylation degree.

The amount of acid necessary for the dissolution of the chitosan is calculated, dependent on the deacetylation degree of the chitosan used, as follows:

TABLE 1

| Deacetylation | Mol acid |
|---|---|
| degree chitosan in % | per g chitosan |
| 80% | 0.00493 |
| 85% | 0.00525 |
| 90% | 0.00555 |
| 95% | 0.00586 |
| 100% | 0.00617 |

For the preparation of the chitosan solution there are added the above described necessary amounts of acid and water to the respective amount of chitosan under stirring, and the ingredients are then stirred until a clear solution is formed.

The chitosan solution obtained in this way may be contacted with an initially moist regenerated cellulose fiber fleece, which is adapted to a defined moisture of 50% to 500% by means of pressing. The fiber fleece may e.g. be soaked by means of spraying. For this reason, in plants for the production of viscose fibers and Modal fibers the so-called bleach field may be used, without the necessity of restructuring the existent production plants.

After impregnation, the fiber fleece may be pressed to a defined moisture of 50%-500%, and subsequently the pressed treatment liquor may be returned into the impregnation cycle.

Thereafter, the fiber fleece will either be treated with hot vapor and subsequently neutrally washed, or it will be neutrally washed without hot vapor treatment, lubricated and dried.

The determination of the amount of chitosan coating is carried out by means of measuring the nitrogen content using the LECO FP 328 nitrogen analyzer by burning up the sample. By means of FITC (fluorescein-isothiocyanate) staining of the fibers and subsequent examination of the fibers using the fluorescence microscope, the chitosan distribution on the fiber surface may be observed.

In another preferred embodiment, the molded body will be subjected to the treatment with a crosslinking agent before or after the drying step.

The present invention in addition relates to a molded body, which is obtainable by the process according to the invention.

The molded body according to the invention has a chitosan content with the above defined specifications, wherein the chitosan is essentially completely distributed at the surface of the molded body (and not in an essential amount also inside the molded body).

The molded body according to the invention may be present particularly in the form of fibers, preferably Lyocell fibers, Modal fibers, polynosic fibers and/or viscose fibers.

One feature of the molded body obtainable by the process according to the invention is that the chitosan is film-like distributed on the surface of the molded body.

The molded body according to the invention has preferably a chitosan content of 0.1 w % and more, preferably 0.2 w % to 1 w %, particularly preferably 0.4 to 0.6 w %. It has been shown that there is obtained in particular a good antibacterial effect of the molded body according to the invention already at small amounts of coating starting at 0.1 w %.

The present invention also relates to the use of a molded body according to the invention as an antibacterial product, as odour-reducing product, as wound healing, styptic and blood coagulation promoting product, in non-woven products and/or as a filling fiber. Preferred areas of use and application of chitosan containing regenerated cellulose fibers according to the invention comprise, due to the mildly antibacterial, odour-reducing and skin-friendly properties, textiles that are worn close to the skin, e.g. underwear or socks, textiles for individuals with sensitive skin (neurodermatitis), bed lining and homewear goods. As a filling fiber, the fiber according to the invention may be used alone or also in mixtures with other fibers, e.g. cotton, polyester fibers and non-modified cellulose fibers (e.g. Lyocell fibers).

In particular there was found out that regenerated cellulose fibers according to the invention have a significantly antibacterial effect already at an amount of chitosan coating of 0.1% in the Shake Flask Test and that they are cell proliferation promoting in the regenerating epidermis (tested in the porcine ex-vivo wound healing model).

The present invention, hence, relates in another aspect also to a molded body according to the invention, in particular with a chitosan content of 0.2 w % to 1 w % for the specific use as wound healing product, in particular as a product promoting the cell proliferation in the regenerating epidermis.

In the following, the invention is further explained in greater detail by means of nonlimiting examples and the figures.

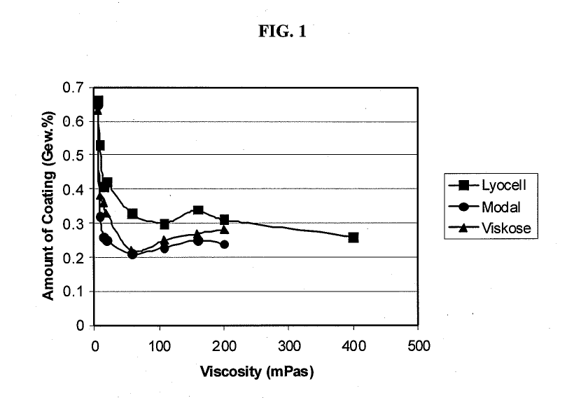

For this reason, FIG. 1 shows the amounts of chitosan coating obtained on various cellulose fibers using the process according to the invention, in dependency of the viscosity of the used chitosan.

### EXAMPLES

### Example 1

There were used the following chitosan types for the treatment of cellulose fibers:

TABLE 2

| | | | Viscosity | Deacetylation |
|---|---|---|---|---|
| Company | Type | Batch n^{o} | mPas | degree % |
| Primex | ChitoClear cg110 | TM2881 | 159 | 82 |
| Primex | ChitoClear cg110 | TM3013 | 108 | 80 |
| Primex | ChitoClear cg110 | TM3089 | 58 | 81 |
| Primex | ChitoClear cg10 | TM2963 | 19 | 81 |
| Primex | ChitoClear fg95LV | TM3091 | 15 | 96 |
| Primex | ChitoClear | TM2875 | 9 | 85 |
| | fg95ULV | | | |
| Heppe | 85/200/A1 | | 200 | 85 |
| Heppe | 85/400/A1 | | 400 | 85 |
| Heppe | 90/10/A1 | | 6 | 90 |

From these chitosan types, there was prepared respectively a 1 w % chitosan solution in aqueous lactic acid. The respective amount of lactic acid used was defined according to the above table 1 in dependency on the deacetylation degree of the used chitosan.

Fiber Samples Used:

1.3 dtex Lyocell fiber, NMMO-free washed, never dried

1.3 dtex Modal fiber, not bleached, never dried

1.3 dtex viscose fiber, not bleached, never dried

Approach for the fiber treatment:

The never-dried fibers were impregnated with the respective chitosan solution for 5 minutes at room temperature in a liquor ratio of 1:10, pressed with 1 bar, then steamed for 5 minutes at 100° C./100% relative moisture, washed and dried at 60° C.

The results of the tests are summarized in the following table.

TABLE 3

| | Amount of | | |
|---|---|---|---|
| Chitosan type | chitosan coating in w % | | |
| Batch N^{o}/Type | Lyocell | Modal | Viscose |
| TM2881 | 0.34 | 0.25 | 0.27 |
| TM3013 | 0.30 | 0.23 | 0.25 |
| TM3089 | 0.33 | 0.21 | 0.22 |
| TM2963 | 0.42 | 0.25 | 0.33 |
| TM3091 | 0.41 | 0.26 | 0.36 |
| TM2875 | 0.53 | 0.32 | 0.38 |
| 85/200/A1 | 0.31 | 0.24 | 0.28 |
| 85/400/A1 | 0.26 | — | — |
| 90/10/A1 | 0.66 | 0.65 | 0.63 |

All fiber samples thus prepared were subjected to hot water treatment in a liquor ratio of 1:20 for 40 minutes at 90° C. The chitosan coatings were shown to be permanent.

The determination of the amount of chitosan coating on the fiber is carried out by measuring the N content (LECO FP 328 nitrogen analyzer) through burning the sample.

A FITC (fluorescein-isothiocyanate) staining of the fibers and subsequent examination of the fibers using the fluorescence microscope were performed in order to analyze the chitosan distribution on the fiber surface.

From the following table, there may be clearly seen for all three fiber types that—in the case of the same w % concentration of chitosan—the lower the viscosity of the solution is, the higher the obtained amount of chitosan coating o will be. Furthermore, the highest amounts of chitosan coating will be obtained at the Lyocell fiber, as these have evidently a better accessible pore system in their initially moist state.

TABLE 4

| Viscosity of the | Amount of | | |
|---|---|---|---|
| chitosan solution | chitosan coating in w % | | |
| 1% in acetic acid mPas | Lyocell | Modal | Viscose |
| 400 | 0.26 | Not tested | Not tested |
| 200 | 0.31 | 0.24 | 0.28 |
| 159 | 0.34 | 0.25 | 0.27 |
| 108 | 0.30 | 0.23 | 0.25 |
| 58 | 0.33 | 0.21 | 0.22 |
| 19 | 0.42 | 0.25 | 0.33 |
| 15 | 0.41 | 0.26 | 0.36 |
| 9 | 0.53 | 0.32 | 0.38 |
| 6 | 0.66 | 0.42 | 0.47 |

This correlation is presented in a diagram in FIG. 1.

### Example 2

### Preparation of a Lyocell Fiber in a Production Test

There was treated a Lyocell fiber with a titer of 1.3 dtex and 38 mm cutting length with chitosan.

The never-dried Lyocell fiber prepared according to the teaching in WO 93/19230 was impregnated according to the process described in example 1 with 1 w % chitosan solution in lactic acid (chitosan type: TM2875, see table 1) in a liquor ratio of 1:20 for an intended amount of coating of 0.4 w % chitosan, vapored, lubricated and dried. From the thus prepared fibers, there was spun yarn n° 50 and processed into a textile fabric (Single jersey knitted fabric), which was shown to have an amount of chitosan coating of 0.45%.

These knitted samples were, in comparison to bleached cotton and Lyocell fibers not treated with chitosan, tested in the porcine ex-vivo wound healing model in the University Hospital Hamburg Eppendorf, Cell-Biological Laboratories, on the portion of proliferative cells at the wound edge in the regenerating epidermis and the part of the epidermis not involved. There were found significantly more proliferative cells at the wound edge and in tendency also more proliferative cells in the regenerating epidermis of the chitosan containing fiber than in a not-treated Lyocell fiber and cotton.

1. A process for the treatment of a cellulosic molded body, wherein the molded body is contacted with an acid solution of a chitosan, and wherein chitosan has a deacetylation degree of at least 80%, a nitrogen content of at least 7 w %, preferably at least 7.5 w %, an average molecular weight M_{w} (D) of 10 kDa to 1000 kDa, and a viscosity in 1 w % solution in 1 w % acetic acid at 25° C. of 1000 mPas or less.

2. The process according to claim 1, wherein the chitosan content in the solution is 0.1 to 10 w %, preferably 1 to 4 w %.

3. The process according to claim 1, wherein the molded body, is present in the form of fibers.

4. The process according to claim 3, wherein the fibers are selected from the group consisting of Lyocell fibers Modal fibers, polynosic fibers viscose fibers, and combinations thereof.

5. The process according to claim 4, wherein the fibers intended for treatment are provided in already dried form as an integral part of a textile article.

6. The process according to claim 4, wherein the fibers are present in never-dried form during the treatment.

7. The process according to claim 6, wherein the fibers are present in the form of a fiber fleece.

8. The process according to claim 6, wherein the fibers have a residual moisture of 50% to 500% before treatment.

9. The process according to any of the previous claims, wherein the molded body is subjected to a treatment with hot vapor after treatment with the solution.

10. The process according to claim 1, wherein in order to prepare the solution, chitosan is dissolved in an inorganic or organic acid.

11. The process according to claim 10, wherein the acid is selected from the group consisting of mono-, di- or tricarboxylic acids with 1 to 30 C-atoms, preferably lactic acid, acetic acid, formic acid, propionic acid, glycolic acid, citric acid, oxalic acid, and mixtures thereof.

12. A molded body obtainable by a process according to claim 1, 2, 3, 4, 5, 6, 7, 8, 9, 10 or 11.

13. The molded body according to claim 12 in a form of fibers.

14. The molded body according to any of the claim 12, having by a chitosan content of 0.1 w %.

15. The molded body according to claim 12, wherein said molded body is selected from the group consisting of an antibacterial product, an odor-reducing product, a wound healing product, a styptic and blood coagulation promoting product, a non-woven product and a filling fiber.

16. The molded body according to claim 23, wherein said molded body is selected from a wound healing product and a product promoting the cell proliferation in the regenerating epidermis.

17. The process according to claim 1, wherein the nitrogen content is at least 7.5 w %.

18. The process according to claim 1, wherein the average molecular weight M_{w} (D) is 10 kDa to 160 kDa.

19. The process according to claim 1, wherein the viscosity in 1 w % solution in 1 w % acetic acid at 25° C. is 400 mPas or less.

20. The process according to claim 1, wherein the viscosity in 1 w % solution in 1 w % acetic acid at 25° C. is 200 mPas or less.

21. The process according to claim 5, wherein the textile article is selected from the group consisting of a yarn, a fabric, a knitted fabric, a piece of clothing produced therewith or a non-woven article.

22. The molded body according to claim 13, wherein the fibers are selected from the group selected from Lyocell fibers, Modal fibers, polynosic fibers, viscose fibers, and mixtures thereof.

23. The molded body according to claim 14, wherein the chitosan content is 0.2 w % to 1 w %.

24. The molded body according to claim 23, wherein the chitosan content is 0.4 w % to 0.6 w %.
